(19) United States
(12) Patent Application Publication
(10) Pub. No.: US 2022/0106564 A1
(43) Pub. Date: Apr. 7, 2022

(54) COMPOSITION AND KIT FOR DIFFERENTIATION OF STEM CELLS INTO NEURAL CREST STEM CELLS, INCLUDING INHIBITOR OF TGF-BETA I RECEPTOR AND BMP INHIBITOR, AND METHOD USING SAME

(71) Applicant: CHA UNIVERSITY INDUSTRY-ACADEMIC COOPERATION FOUNDATION, Seongnam-si (KR)

(72) Inventor: Dong Youn HWANG, Seongnam-si (KR)

(73) Assignee: CHA UNIVERSITY INDUSTRY-ACADEMIC COOPERATION FOUNDATION, Pocheon-si (KR)

(21) Appl. No.: 17/418,980

(22) PCT Filed: Jan. 10, 2020
(86) PCT No.: PCT/KR2020/000437

§ 371 Date: Jun. 28, 2021

(30) Foreign Application Priority Data
Jan. 11, 2019 (KR) 10-2019-0003946

(51) Int. Cl. C12N 5/0797

(52) U.S. Cl. CPC C12N 5/0623; C12N 2506/45; C12N 2506/02; C12N 2501/15; C12N 2501/155

(57) ABSTRACT

Provided are a composition and a kit for differentiation of stem cells into neural crest stem cells (NCSCs), each including a transforming growth factor beta (TGF-β) I receptor inhibitor and 0.001 μM to 2.5 μM of a bone morphogenetic protein (BMP) inhibitor, and a method using the same. Accordingly, stem cells may be efficiently differentiated into neural crest stem cells using the simple, low-cost composition.

### TECHNICAL FIELD

The present disclosure relates to a composition and a kit, each for differentiation of stem cells into neural crest stem cells (NCSCs), and a method using the same.

### BACKGROUND ART

Nervous system cells may be largely divided into two types, central nervous system cells constituting the brain and spinal cord, and peripheral nervous system cells constituting the motor, sensory, and autonomic nerves. Neurons, astrocytes, and oligodendrocytes that constitute the central nervous system (brain and spinal cord) may be produced by differentiating neural stem cells or neural progenitor cells (NPCs) which are differentiated from pluripotent stem cells, whereas the peripheral nerve cells (motor nerve, autonomic nerve, and sensory nerve) and Schwann cells that constitute the peripheral nervous system are derived from neural crest stem cells (NCSCs) which are differentiated from pluripotent stem cells. Therefore, central nervous system cells and peripheral nervous system cells are produced from pluripotent stem cells according to different differentiation pathways, through neural progenitor cells and neural crest stem cells, respectively, and these different pathways are known to depend on the surrounding environment and intracellular signaling systems.

There are about 360,000 people suffering from various peripheral nervous system diseases caused by death of peripheral nervous system cells in the United States alone, costing about 150 trillion dollars annually. The peripheral nervous system diseases impose a great socio-economic burden on society, and therefore, efforts to treat the diseases are urgently needed.

Accordingly, to develop a cell therapy method that is considered as the most fundamental treatment for peripheral nervous system diseases, most of which are refractory and progressive diseases, it is essential and very important to establish a method of efficiently differentiating stem cells into neural crest stem cells, which are parent cells of the peripheral nervous system cells.

### DESCRIPTION OF EMBODIMENTS

### Technical Problem

Provided is a composition for differentiation of stem cells into neural crest stem cells.

Provided is a kit for differentiation of stem cells into neural crest stem cells.

Provided is a method of differentiating stem cells into neural crest stem cells.

### Solution to Problem

An aspect provides a composition for differentiation of stem cells into neural crest stem cells (NCSCs), the composition including a transforming growth factor beta (TGF-β) I receptor inhibitor and 0.001 μM to 2.5 μM of a bone morphogenetic protein (BMP) inhibitor.

The term “TGF-β I receptor” refers to a serine/threonine receptor belonging to the TGF-β receptor family. TGF-β I receptor may also be called TGFBR1, AAT5, ACVRLK4, ALK-5, ALK5, ESS1, LDS1, LDS1A, LDS2A, MSSE, SKR4, TGFR-1, tbetaR-I, TBRI, or TBR-i.

The TGF-β I receptor inhibitor may be a low-molecular-weight compound or polypeptide that inhibits cellular signaling of the TGF-β I receptor. The TGF-β I receptor inhibitor may be, for example, any one or more selected from SB431542 (4-[4-(1,3-benzodioxol-5-yl)-5-(2-pyridinyl)-1H-im idazol-2-yl]benzam ide); SB525334 (6-[2-tert-butyl-5-(6-methyl-pyridin-2-yl)-1H-imidazol-4-yl]-quinoxaline); SB505124 (2-(4-(benzo[d][1,3]dioxol-5-yl)-2-tert-butyl-1H-im idazol-5-yl)-6-methylpyridine); Galunisertib (LY2157299) (4-[2-(6-methylpyridin-2-yl)-5,6-dihydro-4H-pyrrolo[1,2-b]pyrazol-3-yl]quinoline-6-carboxamide); GW788388 (4-(4-(3-(pyridin-2-yl)-1H-pyrazol-4-yl)pyridin-2-yl)-N-(tetrahydro-2H-pyran-4-yl)benzamide); LY2109761 (4-(2-((4-(2-(pyridin-2-yl)-5,6-dihydro-4H-pyrrolo[1,2-b]pyrazol-3-yl)quinolin-7-yl)oxy)ethyl)morpholine); RepSox (2-(3-(6-methylpyridin-2-yl)-1H-pyrazol-4-yl)-1,5-naphthyridine); A 77-01 (4-[3-(6-methyl-2-pyridinyl)-1H-pyrazol-4-yl]-quinoline), and SD-208 (2-(5-chloro-2-fluorophenyl)pteridin-4-yl]pyridin-4-yl-amine).

The term “BMP” refers to a growth factor that induces formation of bone and cartilage. The BMP may be selected from BMP1, BMP2, BMP3, BMP4, BMP5, BMP6, BMP7, BMP8a, BMP8b, BMP10, BMP11, and BMP15.

The BMP inhibitor may be a low-molecular-weight compound or polypeptide that inhibits cellular signaling of BMP. The BMP inhibitor may be a BMP receptor inhibitor, for example, a BMP receptor type 1 inhibitor or a BMP receptor type 2 inhibitor. The BMP inhibitor may be a low-molecular-weight inhibitor or a polypeptide inhibitor. The BMP inhibitor may be selected from dorsomorphin ((6-[4-[2-(1-Piperidinyl)pethoxy]phenyl]-3-(4-pyridinyl)-pyrazolo[1,5-a]pyrimidine); dorsomorphin homolog 1 (DMH1, 4-[6-[4-(1-Methylethoxy)phenyl]pyrazolo[1,5-a]pyrimidin-3-yl]-quinoline); K 02288(3-[(6-amino-5-(3,4,5-trimethoxyphenyl)-3-pyridinyl]phenol); LDN 212854(5-(6-(4-(1-Piperazinyl)phenyl)pyrazolo[1,5-a]pyrim idin-3-yl)quinolone); and Noggin polypeptide. The dorsomorphin may also be called Compound C or BML-275.

The composition may further include a cell culture medium selected from a Dulbecco Modified Eagle's Medium (DMEM), a DMEM/F12, a F-10 nutrient medium (Nutrient M), a minimum essential medium (MEM), an RPMI medium 1640, an Opti-MEM I reduced serum medium, an Iscove's Modified Dulbecco's Medium (IMDM), an alpha-MEM, and a neurobasal medium. The composition may further include one or more selected from a Ham's F12 nutrient mixture, a B27 supplement, an F-10 nutrient mixture, an F-12 nutrient mixture, an N2 supplement, an HT supplement, a G-5 supplement, a lipid supplement, a serum replacement, non-essential amino acids, 13-mercaptoethanol, and an insulin-transferrin-selenium (ITS) supplement.

A concentration of the TGF-β I receptor inhibitor in the composition may be about 0.1 μM to about 100 μM, about 0.5 μM to about 90 μM, about 1 μM to about 80 μM, about 2 μM to about 70 μM, about 3 μM to about 60 μM, about 4 μM to about 50 μM, about 5 μM to about 40 μM, about 6 μM to about 30 μM, about 7 μM to about 20 μM, about 8 μM to about 10 μM, or about 9 μM to about 10 μM.

A concentration of the BMP inhibitor in the composition may be about 0.001 μM to about 2.5 μM, about 0.001 μM to about 2 μM, about 0.001 μM to about 1.5 μM, about 0.001 μM to about 1 μM, about 0.001 μM to less than about 1 μM, about 0.01 μM to about 2.5 μM, about 0.01 μM to about 2.5 μM, about 0.01 μM to about 2 μM, about 0.01 μM to about 1.5 μM, about 0.01 μM to about 1 μM, about 0.01 μM to less than about 1 μM, about 0.1 μM to about 2.5 μM, about 0.1 μM to about 2.4 μM, about 0.1 μM to about 2.3 μM, about 0.1 μM to about 2.2 μM, about 0.1 μM to about 2.1 μM, about 0.1 μM to about 2.0 μM, about 0.1 μM to about 1.9 μM, about 0.1 μM to about 1.8 μM, about 0.1 μM to about 1.7 μM, about 0.1 μM to about 1.6 μM, about 0.1 μM to about 1.5 μM, about 0.1 μM to about 1.4 μM, about 0.1 μM to about 1.3 μM, about 0.1 μM to about 1.2 μM, about 0.1 μM to about 1.1 μM, about 0.1 μM to about 1.0 μM, about 0.1 μM to less than about 1.0 μM, about 0.1 μM to about 0.9 μM, about 0.1 μM to about 0.8 μM, about 0.1 μM to about 0.7 μM, about 0.1 μM to about 0.6 μM, about 0.1 μM to about 0.5 μM, about 0.1 μM to about 1 μM, about 0.2 μM to about 1 μM, about 0.3 μM to about 1 μM, about 0.4 μM to about 1 μM, about 0.5 μM to about 1 μM, or about 0.1 μM to less than about 1 μM.

The composition may include a cell culture medium, a serum replacement, a TGF-β I receptor inhibitor, and 0.001 μM to 1 μM of DMH1. The composition may include a cell culture medium, a serum replacement, a TGF-β I receptor inhibitor, and 0.001 μM to less than 1 μM of dorsomorphin.

The concentrations of the components in the composition may be the final concentrations. The composition may be concentrated, dried, or diluted. For example, when the composition is concentrated 50-fold, the composition may be added to a medium including stem cells at a final concentration diluted 1/50.

The composition may be a composition for cell culture.

The term “stem cells” refer to totipotent cells that are able to differentiate into all types of cells or pluripotent cells that are able to differentiate into various types of cells, and stem cells which are undifferentiated cells may differentiate into cells of a specific tissue. The stem cells may be embryonic stem cells (ESCs), adult stem cells, induced pluripotent stem cells (iPSC)s, or somatic cell nuclear transfer embryonic stem cells. The embryonic stem cells refer to an in vitro culture of an extract of an inner cell mass of a blastocyst, which is formed immediately before implantation of a fertilized egg in the mother's uterus. The adult stem cells, which are undifferentiated cells that exist only in a small amount in each tissue of the body, refer to cells that replace dead or damaged tissue. The induced pluripotent stem cells (iPSCs) refer to cells that are induced to have pluripotency like embryonic stem cells by injecting cell dedifferentiation-related genes into somatic cells that have undergone differentiation and returning the cells to an early stage of totipotent stem cells. The induced pluripotent stem cells may be, for example, human dermal fibroblast-derived induced pluripotent stem cells (human dermal fibroblast-iPSCs: hDF-iPSCs), blood cell-derived induced pluripotent stem cells (blood cell-iPSCs), or urine cell-derived induced pluripotent stem (urine-iPSCs). The somatic cell nuclear transfer embryonic stem cells refer to totipotent cells obtained from an in vitro culture of an extract of an inner cell mass of a blastocyst, which is formed during the initial development of cells prepared by removing a nucleus of an egg and replacing the nucleus with a nucleus of a somatic cell.

The stem cells may be cells derived from a mammal, for example, a human, mouse, rat, ape, cow, horse, pig, dog, sheep, goat, or cat.

The term “neural crest” refers to a sprouting population of ectodermal cells which are derived from the belly ectoderm of a vertebrate and located above the neural tube along the midline underneath the dorsal ectoderm. The neural crest is often observed as a tubular form on the neural tube in a transverse section, and generated by deepithelialization of a population of cells at the outer edge of the neural fold. As the neural crest develops, it divides into left and right halves, and descends by passing through the edge of the neural tube, and migrates widely in the embryo to differentiate into nerve ganglia, mesenchyme, head, branchial arch skeleton, and pigment cells. Neural crest stem cells (NCSCs) may be cells that appear inside the embryo at the adhesion site when two neural folds of the left and right sides adhere to each other to deepithelialize. Depending on their location, the neural crest stem cells may be largely divided into cranial neural crest stem cells, cardiac neural crest stem cells, trunk neural crest stem cells, and vagal and sacral neural crest stem cells.

The neural crest stem cells may express P75 protein, HNK1, or a combination thereof. The neural crest stem cells may further express SOX10, AP2, FoxD3, NOTCH1, SNAIL, vinculin, bone morphogenetic protein (BMP)-4, BMP-7, or a combination thereof. The neural crest stem cells may not express SOX1. SOX1 may be expressed in neural precursor cells (NPCs) which are progenitors of the central nervous system cells.

The term “differentiation” refers to a phenomenon in which a cell's structure or function is specialized during growth by cell division and proliferation. A totipotent or pluripotent stem cell may completely differentiate into a specific cell via a specific type of progenitor cell. The embryonic stem cells, induced pluripotent stem cells, or nuclear transfer embryonic stem cells may differentiate into the neural crest stem cells. The neural crest stem cells may differentiate into peripheral nerve cells, Schwann cells, melanocytes, osteocytes, chondrocytes, muscle cells, etc.

The composition may be a single composition or a separate composition.

Another aspect provides a kit for differentiation of stem cells into neural crest stem cells, the kit including the composition according to one aspect and a cell culture dish.

The composition, the stem cells, the neural crest stem cells, and the differentiation are the same as described above.

The cell culture dish refers to a cell culture vessel, and includes any cell culture vessel regardless of the material, size, and shape of the culture dish.

The cell culture dish may be a culture dish for suspension culture or a culture dish for adherent culture. The culture dish for adherent culture may be coated with a polypeptide. The polypeptide may be a polypeptide for adhering or culturing stem cells. The polypeptide is, for example, vitronectin (VTN), laminine, fibronectin, poly ornithine, or Matrigel™.

Still another aspect provides a method of differentiating stem cells into neural crest stem cells, the method including suspension-culturing the stem cells in a cell culture medium containing a TGF-β I receptor inhibitor and 0.001 μM to 2.5 μM of a BMP inhibitor to obtain embryoid bodies (EBs); and adherent-culturing cells which are obtained by chopping the EBs to induce differentiation into neural crest stem cells.

The stem cells, the TGF-β I receptor, the TGF-β I receptor inhibitor, the BMP, the BMP inhibitor, the cell culture medium, the neural crest stem cells, and the differentiation are the same as described above.

The method may further include inoculating the stem cells in the cell culture dish. The stem cells may be inoculated in the presence of a basic culture medium.

The method includes suspension-culturing the stem cells in a cell culture medium containing a TGF-β I receptor inhibitor and 0.001 μM to 2.5 μM of a BMP inhibitor to obtain EBs.

The suspension-culturing may be culturing without adhering the stem cells onto the bottom of the culture dish. When the stem cells are suspension-cultured, an EB may be formed, which is a cell mass formed by aggregation of stem cells into a ball shape at the beginning of cell division.

In the method, the culture time of the stem cells may vary depending on the culture conditions. The stem cells may be cultured for, for example, about 1 day to about 10 days, about 1 day to about 9 days, about 1 day to about 8 days, about 1 day to about 7 days, about 1 day to about 6 days, about 1 day to about 5 days, about 1 day to about 4 days, about 2 day to about 4 days, about 3 day to about 4 days, or about 4 days. In the method, the stem cells may be cultured at about 30° C. to about 40° C., about 30° C. to about 37° C., or about 37° C.

The method includes adherent-culturing cells which are obtained by chopping the EBs to induce differentiation into neural crest stem cells.

The adherent culturing may be culturing by adhering cells onto the bottom of the culture dish.

The adherent culturing may be performed for about 1 day to about 15 days, about 1 day to about 14 days, about 1 day to about 13 days, about 1 day to about 12 days, about 1 day to about 11 days, about 1 day to about 10 days, about 1 day to about 9 days, about 1 day to about 8 days, about 1 day to about 7 days, about 1 day to about 6 days, about 1 day to about 5 days, or about 2 days to about 5 days.

A proportion of neural crest stem cells in the cells cultured by the method may be about 50% or more, about 60% or more, about 70% or more, about 80% or more, about 85% or more, about 90% or more, about 95% or more, about 97% or more, about 98% or more, or about 99% or more.

### Advantageous Effects of Disclosure

According to a composition and a kit for differentiation of stem cells into neural crest stem cells (NCSCs), each including a TGF-β I receptor inhibitor and 0.001 μM to 2.5 μM of a BMP inhibitor, and a method using the same, stem cells may be efficiently differentiated into neural crest stem cells using the simple, low-cost composition.

### BRIEF DESCRIPTION OF DRAWINGS

FIG. 1A shows an illustration of a process of inducing differentiation of human embryonic stem cells, FIG. 1B shows microscopic images of embryoid bodies which were obtained by suspension-culturing human embryonic stem cells for about 4 days (upper: 10 μM of SB431542 and various concentrations of dorsomorphin, lower: 10 μM of SB431542 and various concentrations of DMH1), FIG. 1C shows microscopic images of cells which were obtained by suspension-culturing for about 4 days and adherent-culturing for about 5 days (upper: 10 μM of SB431542 and various concentrations of dorsomorphin, lower: 10 μM of SB431542 and various concentrations of DMH1, black arrows: cells forming rosettes, white arrows: embryoid body), and FIG. 1D shows a graph showing the number of embryoid bodies per unit area (embryoid body/cm²) according to DMH1 concentrations (***: p<0.001);

FIG. 2A shows graphs showing results of flow cytometry for detecting P75 in cultured cells, and FIG. 2B shows graphs showing results of flow cytometry for detecting SOX1 in cultured cells(upper: SB431542 and dorsomorphin, lower: SB431542 and DMH1); and

FIG. 3A shows microscopic images of embryoid bodies which were obtained by suspension-culturing human embryonic stem cells in the presence of 10 μM of SB431542 and 0.5 μM to 2.5 μM of DMH1, FIG. 3B shows microscopic images of embryoid bodies which were obtained by suspension-culturing human embryonic stem cells for about 4 days in the presence of 10 μM of SB431542 and 0.5 μM to 2.5 μM of dorsomorphin, FIG. 3C shows microscopic images of cells which were obtained by suspension-culturing and adherent-culturing in the presence of 10 μM of SB431542 and 0.5 μM to 2.5 μM of DMH1 (black arrows: cells forming rosettes, white arrows: embryoid body), FIG. 3D shows microscopic images of cells which were obtained by suspension-culturing and adherent-culturing in the presence of 10 μM of SB431542 and 0.5 μM to 2.5 μM of dorsomorphin, and FIG. 3E shows graphs showing results of flow cytometry for detecting P75 in cultured cells (upper: SB431542 and DMH1, lower: SB431542 and dorsomorphin).

### MODE OF DISCLOSURE

Hereinafter, the present disclosure will be described in more detail with reference to exemplary embodiments. However, these exemplary embodiments are only for illustrating the present disclosure, and the scope of the present disclosure is not limited to these exemplary embodiments.

### EXAMPLE 1

### Differentiation of Human Pluripotent Stem Cells into Neural Crest Stem Cells

1. Induction of differentiation into neural crest stem cells

Colonies obtained by culturing H9 human embryonic stem cells (hESCs) (WiCell Research Institute, Inc. Madison, Wis., U.S.A.) in a culture medium were dissociated into single cells using accutase (Life Technologies).

The cells dissociated into single cells were seeded in a culture plate coated with a protein such as vitronectin (VTN) (vitronectin XF, STEMCELL Technologies), lam inine (rhLaminin-521, Thermo Fisher Scientific Inc., Waltham, Mass., USA), fibronectin (Thermo Fisher Scientific Inc.), or Matrigel™ (StemCell Technologies, Inc.). An embryonic stem cell (ESC) culture medium was added to the seeded cells to perform undifferentiation culture. As the ESC culture medium, Essential 8 (E8) medium (StemCell Technologies Inc.), TeSR2 medium (StemCell Technologies Inc), StemMACS (Miltenyi Biotec, Bergisch Gladbach, Germany), etc. was used.

The obtained colonies were finely chopped and suspension-cultured under conditions of 37° C. and 5% CO₂ in an EB medium containing 10 μM of SB431542 (GlaxoSmithKline: GSK) as a TGF-β I receptor inhibitor and 0.5 μM to 1 μM of dorsomorphin homolog 1 (DMH1) (Tocris Bioscience, USA) or dorsomorphin (Tocris Bioscience, USA) as a BMP inhibitor for about 4 days to prepare embryoid bodies (EBs). EB medium contained DMEM/F12 (Life Technologies), 20%(v/v) Knockout serum replacement (KSR, Invitrogen), lx non-essential amino acids (Invitrogen), 0.1 mM 13-mercaptoethanol (Invitrogen), and lx penicillin/streptomycin (Invitrogen). As a negative control, dimethyl sulfoxide (DMSO) (Sigma-Aldrich) was used instead of the drug.

EBs, which are round cell masses produced by suspension-culture for about 4 days, were finely chopped, and then adhered to the bottom of the culture plate, followed by adherent culture for about 5 days (DMEM-F12, 1× N2 supplement, 25 μg/ml of insulin, 20 ng/ml of bFGF, and a matrigel-coated culture plate were used).

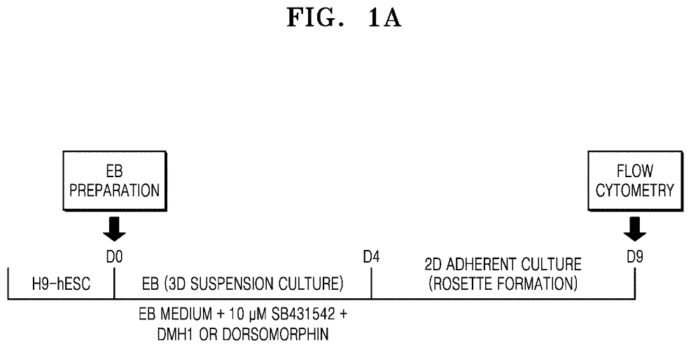

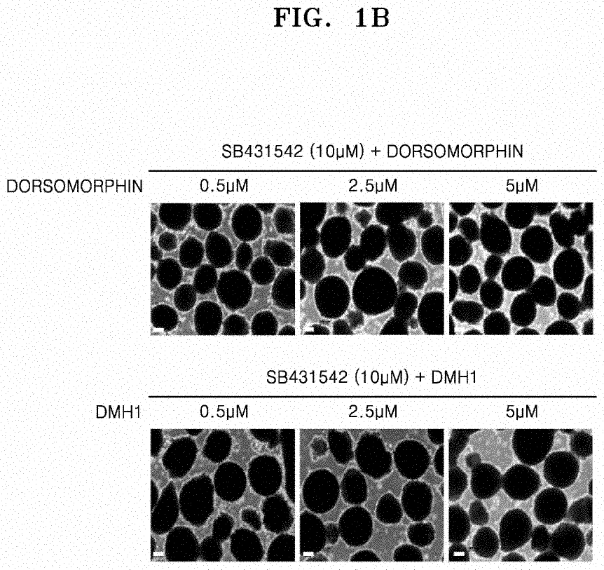

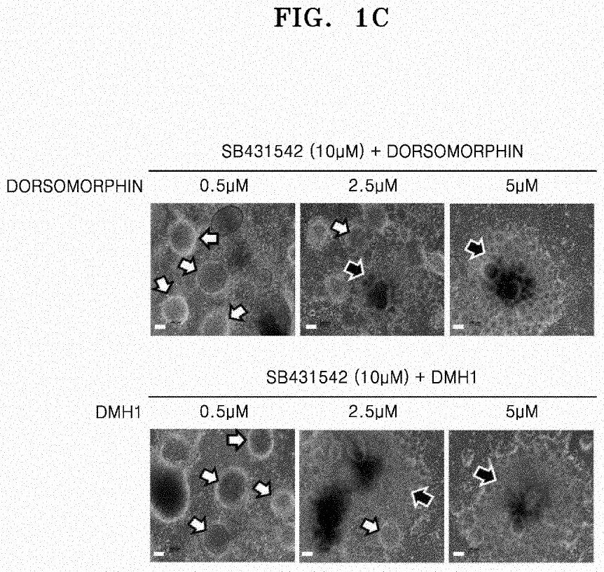

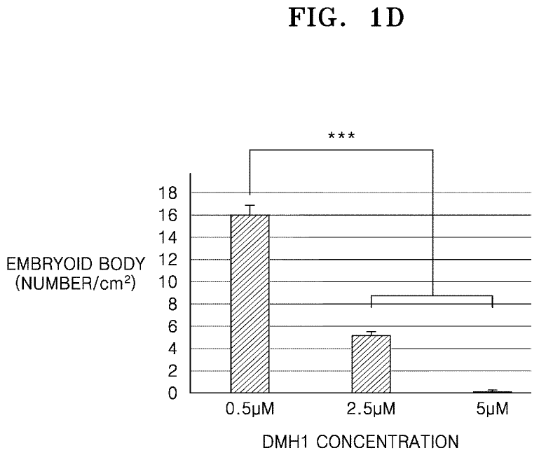

Microscopic images of embryoid bodies which were suspension-cultured for about 4 days are shown in FIG. 1B, and microscopic images of cells forming rosettes, which were obtained by suspension-culture for about 4 days and adherent-culture for about 5 days (upper: 10 μM of SB431542 and various concentrations of dorsomorphin, lower: 10 μM of SB431542 and various concentrations of DMH1, black arrows: cells forming rosettes, white arrows: embryoid body) are shown in FIG. 1C. The number of round-shaped embryoid bodies were counted, and the number of embryoid bodies per unit area (embryoid body/cm²) according to DMH1 concentrations is shown in FIG. 1D (***: p<0.001). As shown in FIG. 1D, the number of embryoid bodies per unit area significantly decreased with increasing concentrations of DMH1 in the EB medium during the suspension-culture.

2. Identification of differentiated cells

To examine whether the cells cultured as described in Example 1.1 were neural crest stem cells, flow cytometry was performed.

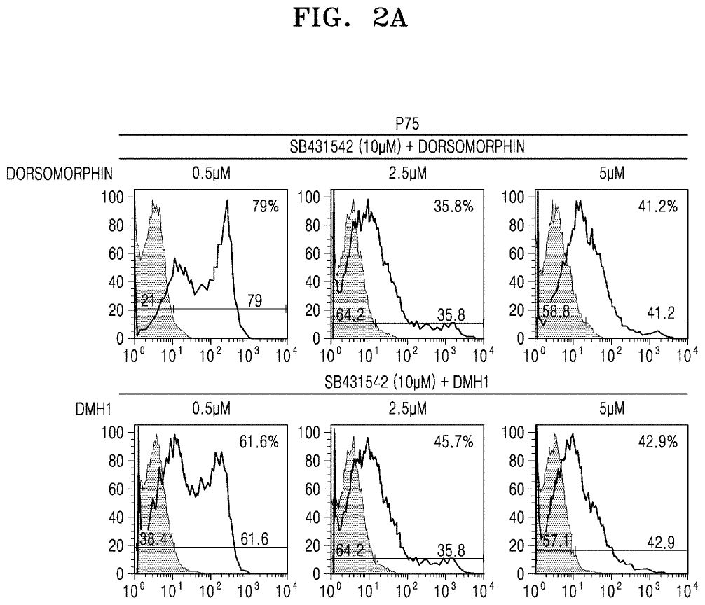

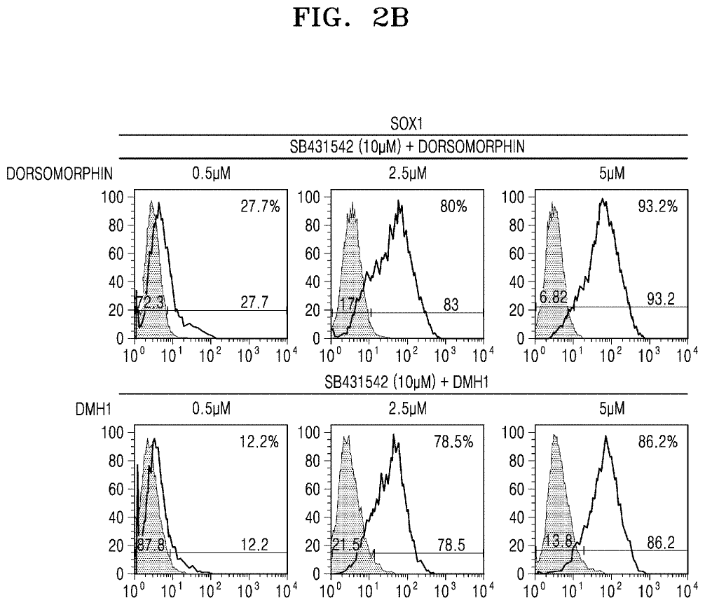

2%(v/v) paraformaldehyde (Sigma-Aldrich) was added to cells cultured for a total of 9 days (suspension culture for 4 days+adherent culture for 5 days), and allowed to react at room temperature for about 10 minutes to fix the cells. 2%(v/v) normal serum/1×PBS (Vector Laboratories, Inc., Burlingame, Calif.) containing 0.1%(v/v) TRITON™ X-100 (Sigma-Aldrich) was added to the cells, and allowed to react at room temperature for 30 minutes to block the fixation. To analyze a proportion of cells expressing P75 protein which is a neural crest stem cell (NCSC) marker, or SOX1 which is a neural progenitor cell (NPC) marker, immunostaining was performed using phycoerythrin (PE)-labeled anti-P75 monoclonal antibody (1:50 dilution) (Miltenyi Biotec), and phycoerythrin (PE)-labeled anti-Sox1 monoclonal antibody (1:100 dilution) (BD Biosciences). The immunostained cells were subjected to flow cytometry using a BD FACSCalibur flow cytometer (BD Biosciences, Sparks, Md., USA). The results of flow cytometry are shown in FIGS. 2A and 2B (FIG. 2A: a graph of P75 flow cytometry, FIG. 2B: a graph of SOX1 flow cytometry).

As shown in FIGS. 2A and 2B, when H9-hESCs were cultured in the EB medium containing 10 μM of SB431542 and dorsomorphin or DMH1, a proportion of P75-expressing cells was about 61% or more in the presence of 0.5 μM of dorsomorphin or DMH1, whereas a proportion of SOX1-expressing cells was about 86% or more in the presence of 5 μM of dorsomorphin or DMH1. Accordingly, when the concentration of the BMP inhibitor such as dorsomorphin or DMH1 was lower, differentiation into P75-expressing cells (i.e., neural crest stem cells) was observed, and when the concentration of dorsomorphin or DMH1 was higher, differentiation into SOX1-expressing cells (i.e., neural progenitor cells) was observed.

3. Culture of embryonic stem cells in the presence of 10 μM of SB431542 and 0.5 μM to 2.5 μM of BMP inhibitor

As the concentration of dorsomorphin or DMH1, which is the BMP inhibitor, was lower, human embryonic stem cells were differentiated into neural crest stem cells. Thus, embryonic stem cells were cultured in the presence of dorsomorphin or DMH1 in the concentration range of 0.5 μM to 2.5 μM, as described in Example 1.1.

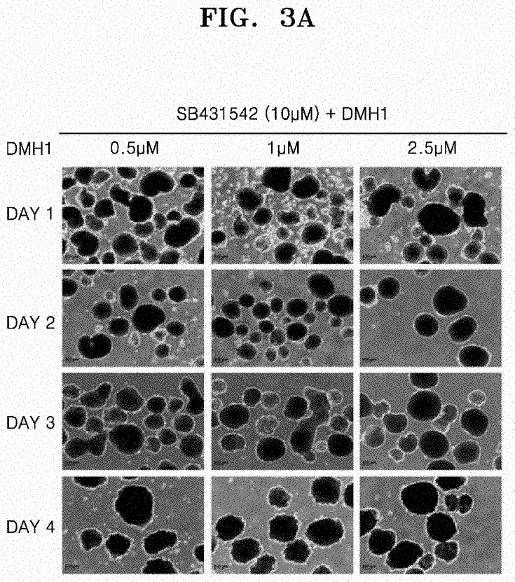

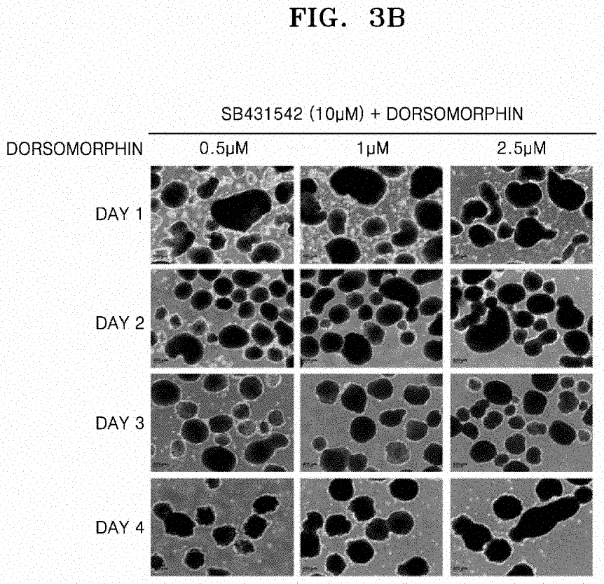

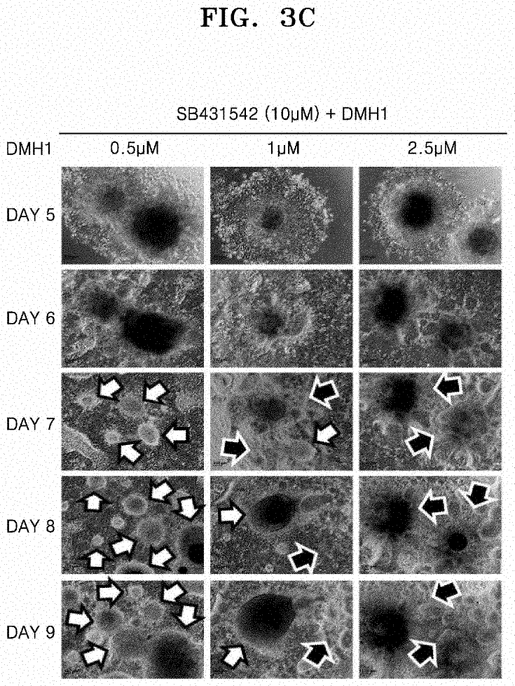

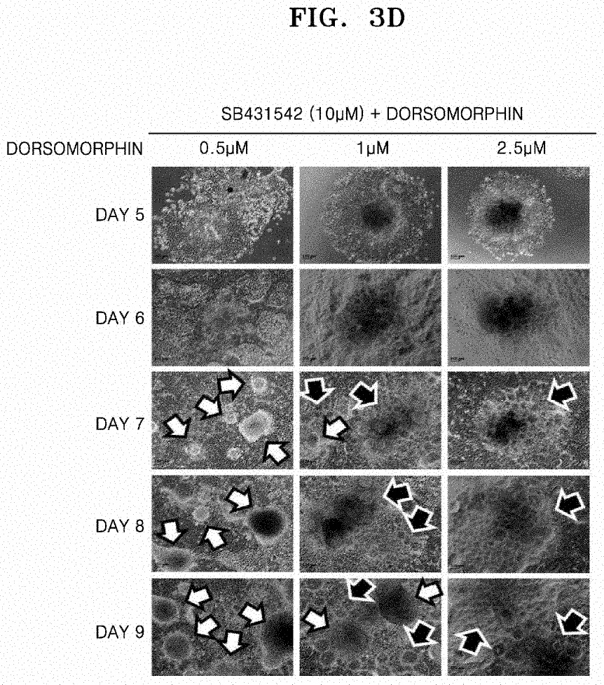

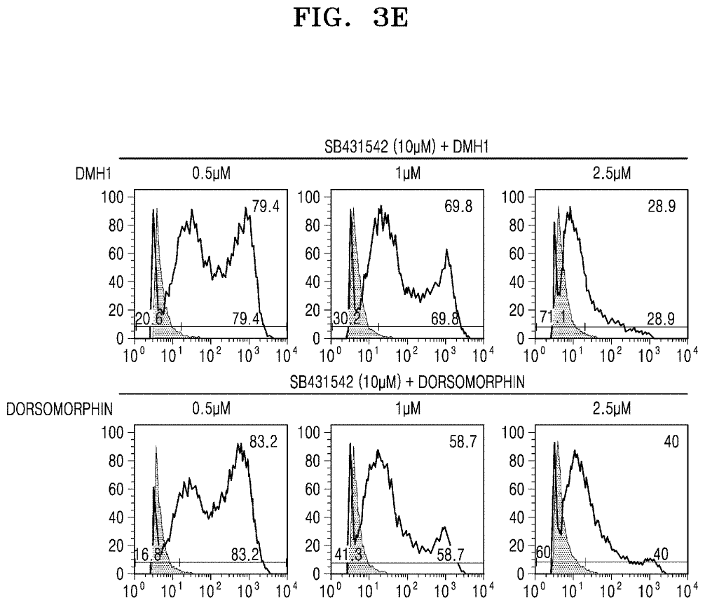

Microscopic images of cells which were obtained by suspension-culturing H9-hESCs in the presence of 10 μM of SB431542 and 0.5 μM to 2.5 M of DMH1 or dorsomorphin are shown in FIGS. 3A and 3B. Further, microscopic images of embryoid bodies obtained by suspension culture for about 4 days are shown in FIGS. 3C and 3D (black arrows: cells forming rosettes, white arrows: embryoid body).

Cells suspension-cultured for about 3 days and adherent-cultured for about 4 days were subjected to P75 flow cytometry, as described in Example 1.2, and the results of flow cytometry are shown in FIG. 3E (upper: SB431542 and DMH1, lower: SB431542 and dorsomorphin).

As shown in FIG. 3E, when H9-hESCs were cultured in the EB medium containing 10 μM of SB431542 and DMH1, a proportion of P75-expressing cells was about 69.8% or more in the presence of 0.5 μM to 1 μM of DMH1, but the proportion of P75-expressing cells showed a remarkable decrease of about 28.9% in the presence of 2.5 μM of DMH1. Further, when H9-hESCs were cultured in the EB medium containing 10 μM of SB431542 and dorsomorphin, a proportion of P75-expressing cells was about 83.2% or more in the presence of 0.5 μM of DMH1, but the proportion of P75-expressing cells showed a decrease of about 58.7% to about 40% in the presence of 1 μM to 2.5 μM of DMH1. Accordingly, it was confirmed that differentiation into neural crest stem cells is induced, as the concentration of the BMP inhibitor such as dorsomorphin or DMH1 is lower.

1. A composition for differentiation of stem cells into neural crest stem cells (NCSCs), the composition comprising a transforming growth factor beta (TGF-β) I receptor inhibitor and 0.001 μM to 2.5 μM of a bone morphogenetic protein (BMP) inhibitor.

2. The composition of claim 1, wherein the TGF-β I receptor inhibitor is one or more selected from
SB431542 (4-[4-(1,3-benzodioxol-5-yl)-5-(2-pyridinyl)-1H-im idazol-2-yl]benzamide);
SB525334 (6-[2-tert-butyl-5-(6-methyl-pyridin-2-yl)-1H-imidazol-4-yl]-quinoxaline);
SB505124 (2-(4-(benzo[d][1,3]dioxol-5-yl)-2-tert-butyl-1H-imidazol-5-yl)-6-methylpyridine);
Galunisertib (LY2157299) (4-[2-(6-methylpyridin-2-yl)-5,6-dihydro-4H-pyrrolo[1,2-b]pyrazol-3-yl]quinoline-6-carboxamide);
GW788388 (4-(4-(3-(pyridin-2-yl)-1H-pyrazol-4-yl)pyridin-2-yl)-N-(tetrahydro-2H-pyran-4-yl)benzamide);
LY2109761 (4-(2-((4-(2-(pyrid in-2-yl)-5, 6-d ihydro-4H-pyrrolo[1, 2-b]pyrazol-3-yl)quinolin-7-yl)oxy)ethyl)morpholine);
RepSox (2-(3-(6-methylpyridin-2-yl)-1H-pyrazol-4-yl)-1,5-naphthyridine);
A 77-01 (4-[3-(6-methyl-2-pyridinyl)-1H-pyrazol-4-yl]-quinoline); and
SD-208 (2-(5-chloro-2-fluorophenyl)pteridin-4-yl]pyridin-4-yl-amine).

3. The composition of claim 1, wherein the BMP inhibitor is one or more selected from
dorsomorphin ((6-[4-[2-(1-piperidinyl)pethoxy]phenyl]-3-(4-pyridinyl)-pyrazolo[1,5-a]pyrimidine);
dorsomorphin homolog 1 (DMH1, 4-[6-[4-(1-methylethoxy)phenyl]pyrazolo[1,5-a]pyrimidin-3-yl]-quinoline);
K 02288(3-[(6-amino-5-(3,4,5-trimethoxyphenyl)-3-pyridinyl]phenol);
LDN 212854(5-(6-(4-(1-piperazinyl)phenyl)pyrazolo[1,5-a]pyrimidin-3-yl)quinolone); and
Noggin polypeptide.

4. The composition of claim 1, further comprising a cell culture medium selected from a Dulbecco Modified Eagle's Medium (DMEM), a DMEM/F12, a F-10 nutrient medium (Nutrient M), a minimum essential medium (MEM), an RPMI medium 1640, an Opti-MEM I reduced serum medium, an Iscove's Modified Dulbecco's Medium (IMDM), an alpha-MEM, and a neurobasal medium.

5. The composition of claim 4, further comprising one or more selected from a Ham's F12 nutrient mixture, a B27 supplement, an F-10 nutrient mixture, an F-12 nutrient mixture, an N2 supplement, an HT supplement, a G-5 supplement, a lipid supplement, a serum replacement, non-essential amino acids, β-mercaptoethanol, and an insulin-transferrin-selenium (ITS) supplement.

6. The composition of claim 1, wherein a concentration of the TGF-β I receptor inhibitor in the composition is 0.1 μM to 100 μM.

7. The composition of claim 1, wherein a concentration of the BMP inhibitor in the composition is 0.001 μM to 1 μM.

8. The composition of claim 1, comprising a cell culture medium, a serum replacement, the TGF-β I receptor inhibitor, and 0.001 μM to 1 μM of DMH1.

9. The composition of claim 1, comprising a cell culture medium, a serum replacement, the TGF-β I receptor inhibitor, and 0.001 μM to less than 1 μM of dorsomorphin.

10. The composition of claim 1, wherein the stem cells are embryonic stem cells (ESCs), adult stem cells, induced pluripotent stem cells (iPSC)s, or somatic cell nuclear transfer embryonic stem cells.

11. A kit for differentiation of stem cells into neural crest stem cells, the kit comprising the composition of claim 1 and a cell culture dish.

12. A method of differentiating stem cells into neural crest stem cells, the method comprising:
suspension-culturing the stem cells in a cell culture medium comprising a TGF-β I receptor inhibitor and 0.001 μM to 2.5 μM of a bone morphogenetic protein (BMP) inhibitor to obtain embryoid bodies (EBs); and
adherent-culturing cells which are obtained by chopping the EBs to induce differentiation into neural crest stem cells.

13. The method of claim 12, wherein the suspension-culturing is performed for 1 day to 10 days.

14. The method of claim 12, wherein the adherent-culturing is performed for 1 day to 15 days.
